(19) United States
(12) Patent Application Publication
(10) Pub. No.: US 2026/0301171 A1
(43) Pub. Date: Oct. 1, 2026

(54) SYSTEM AND METHOD FOR OBJECT VISUALIZATION DURING AN OPHTHALMIC PROCEDURE

(71) Applicant: Alcon Inc., Fribourg (CH)

(72) Inventors: Zhuoran Wu, Rancho Cucamonga, CA (US); Lu Yin, Keller, TX (US); Vignesh Suresh, Woodland Hills, CA (US); Ramesh Sarangapani, Coppell, TX (US); Joseph Weatherbee, San Clemente, CA (US)

(21) Appl. No.: 19/568,045

(22) Filed: Mar. 16, 2026

Related U.S. Application Data
(60) Provisional application No. 63/778,914, filed on Mar. 27, 2025.

(51) Int. Cl. G06T 7/00; A61B 3/00; G06T 3/60

(52) U.S. Cl. CPC G06T 7/0012; A61B 3/0025; G06T 3/60; G06T 2207/10004; G06T 2207/10028; G06T 2207/20081; G06T 2207/20212; G06T 2207/30041

(57) ABSTRACT

A method of augmenting an image from an ophthalmic surgical suite includes receiving an input image from the ophthalmic surgical suite from at least one sensor, identifying an object in the input image, and selecting an object representation corresponding to the object identified in the input image. The method also includes determining a 6D pose estimation for the object in the input image by creating a first set of generated 6D pose images each illustrating the object representation in one of a set of first coordinates, identifying a first match between the first set of generated 6D pose images and the object in the input image, and selecting the 6D pose estimation for the object based on coordinates of the first match. The method also includes augmenting the input image based on the 6D pose estimation.

### INTRODUCTION

This disclosure relates generally to visualizing an object during an ophthalmic procedure and determining a pose for the object.

During ophthalmic procedures, a surgeon may utilize various surgical instruments during the procedure and illuminate areas of interest during the procedure at different illumination levels. A camera system can capture images of the procedure and display the images on a display. During the procedure, the surgeon may be directly manipulating the surgical instruments or through controlling a robotic arm.

### SUMMARY

Disclosed herein is a method of augmenting an image from an ophthalmic surgical suite. The method includes receiving an input image from the ophthalmic surgical suite from at least one sensor, identifying an object in the input image, and selecting an object representation corresponding to the object identified in the input image. The method also includes determining a 6D pose estimation for the object in the input image by creating a first set of generated 6D pose images each illustrating the object representation in one of a set of first coordinates, identifying a first match between the first set of generated 6D pose images and the object in the input image, and selecting the 6D pose estimation for the object based on coordinates of the first match. The method also includes augmenting the input image based on the 6D pose estimation.

In one aspect of the disclosure estimating the 6D pose for the object in the input image includes creating a second set of generated 6D pose images each illustrating the object representation in one of a set of second coordinates with the second coordinates each defining a longitudinal and rotational position for the object representation. Estimating the 6D pose also includes identifying a second match between the second set of generated 6D pose images and the object in the input image with the second match having the least variation from the object and the corresponding one of the second set of generated 6D pose images and updating the 6D pose estimation for the object based on coordinates of the second match.

In one aspect of the disclosure the first set of coordinates includes a first level of coarseness between at least one of longitudinal and rotational positions and the second set of coordinates includes a second level of coarseness between at least one of longitudinal and rotational positions that is less than the first level of coarseness.

In one aspect of the disclosure the first set of generated 6D pose images includes a greater number of images than the second set of generated 6D pose images.

In one aspect of the disclosure the second set of coordinates are within a predetermined range of rotational positions from the first match.

In one aspect of the disclosure the second set of coordinates are within a predetermined range of longitudinal positions from the first match.

In one aspect of the disclosure augmenting the input image includes generating directional markers guiding the object to a desired location within the input image during an ophthalmic procedure.

In one aspect of the disclosure a machine learning model is utilized to identify the first match from the first set of generated 6D pose images with the object identified in the input image.

In one aspect of the disclosure the method includes training the machine learning model to identify an occluded portion of the object in the input image by generating a training dataset including the object representation at a predetermined number of coordinates with known translational and rotational positions and modifying the training dataset to occlude a portion of the object representation from corresponding images in the training dataset to generate a modified training dataset. The method also includes utilizing the modified training dataset with the training dataset as a ground truth in connection with a machine learning algorithm to train the machine learning model to identify the occluded portion of the object in the input image.

In one aspect of the disclosure augmenting the image includes identifying an occluded portion of the object in the input image and highlighting the occluded portion of the object in the input image.

In one aspect of the disclosure highlighting the occluded portion of the object in the input image includes generating an image overlay having a dashed line surrounding a perimeter of the occluded portion.

In one aspect of the disclosure highlighting the occluded portion of the object in the input image includes generating an image overlay of the object representation based on the 6D pose estimation corresponding to a region of the occluded portion.

In one aspect of the disclosure the object representation is generated based on a set of input images illustrating the object in a set of different longitudinal and rotational positions relative to the at least one sensor.

In one aspect of the disclosure the object representation is generated based on a set of input images illustrating an illumination intensity of the object at a set of different levels of intensity.

In one aspect of the disclosure the input image illustrates an ophthalmic surgical suite and the object includes at least one piece of surgical equipment within the ophthalmic surgical suite and the method includes generating a notification if the 6D pose estimation for the at least one piece of surgical equipment is located outside of a predetermined region in the ophthalmic surgical suite.

Disclosed herein is a system for visualizing an object in an ophthalmic surgical suite. The system includes a single optical sensor configured to obtain an input image from the ophthalmic procedure and a controller in communication with the single optical sensor. The controller is configured to receive an input image from the ophthalmic surgical suite from at least one sensor, identify an object in the input image, select an object representation corresponding to the object identified in the input image, and determine a 6D pose estimation for the object in the input image. The 6D pose estimation is determined creating a first set of generated 6D pose images each illustrating the object representation in one of a set of first coordinates, identifying a first match between the first set of generated 6D pose images and the object in the input image, and selecting the 6D pose estimation for the object based on coordinates of the first match. The controller is also configured to augment the input image based on the 6D pose estimation.

In one aspect of the disclosure the controller is configured to determine the 6D pose estimation for the object in the input image by creating a second set of generated 6D pose images each illustrating the object representation in one of a set of second coordinates with the second coordinates each define a longitudinal and rotational position for the object representation and identifying a second match between the second set of generated 6D pose images and the object in the input image. The second match includes the least variation from the object and the corresponding one of the second set of generated 6D pose images. The 6D pose estimation for the object is updated based on coordinates of the second match.

Disclosed herein is a non-transitory computer-readable medium embodying programmed instructions which, when executed by a processor, are operable for performing a method. The method includes receiving an input image from the ophthalmic surgical suite from at least one sensor, identifying an object in the input image, and selecting an object representation corresponding to the object identified in the input image. The method also includes determining a 6D pose estimation for the object in the input image by creating a first set of generated 6D pose images each illustrating the object representation in one of a set of first coordinates, identifying a first match between the first set of generated 6D pose images and the object in the input image, and selecting the 6D pose estimation for the object based on coordinates of the first match. The method also includes augmenting the input image based on the 6D pose estimation.

In one aspect of the disclosure training the machine learning model to identify occluded portions of the object in the input image includes modifying the training dataset to occlude a portion of the object representation from corresponding images in the training dataset to generate a modified training dataset and utilizing the modified training dataset with the training dataset as a ground truth in connection with the machine learning algorithm to train the machine learning model to identify the occluded portion of the object in the input image.

### BRIEF DESCRIPTION OF THE DRAWINGS

FIG. 1 is an illustration of an exemplary ophthalmic surgical suite for performing an ophthalmic procedure.

FIG. 2 is a flow diagram describing an embodiment of a method of augmenting an input image utilizing a six degree-of-freedom (6D) pose estimation of an object during a surgical procedure.

FIG. 3 is a flow diagram describing an embodiment of a method of determining a 6D pose estimation in the method of FIG. 2.

FIG. 4 is an example of an input image augmented by the method of FIG. 2.

FIG. 5 is another example of an input image augmented by the method of FIG. 2.

FIG. 6 is yet another example of an input image augmented by the method of FIG. 2.

FIG. 7 is a further example of an input image augmented by the method of FIG. 2.

FIG. 8 is yet a further example of an input image including a surgical suite augmented by the method of FIG. 2.

The foregoing and other features of the present disclosure are more fully apparent from the following description and appended claims, taken in conjunction with the accompanying drawings.

### DETAILED DESCRIPTION

Embodiments of the present disclosure are described herein. It is to be understood, however, that the disclosed embodiments are merely examples and other embodiments can take various and alternative forms. The figures are not necessarily drawn to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

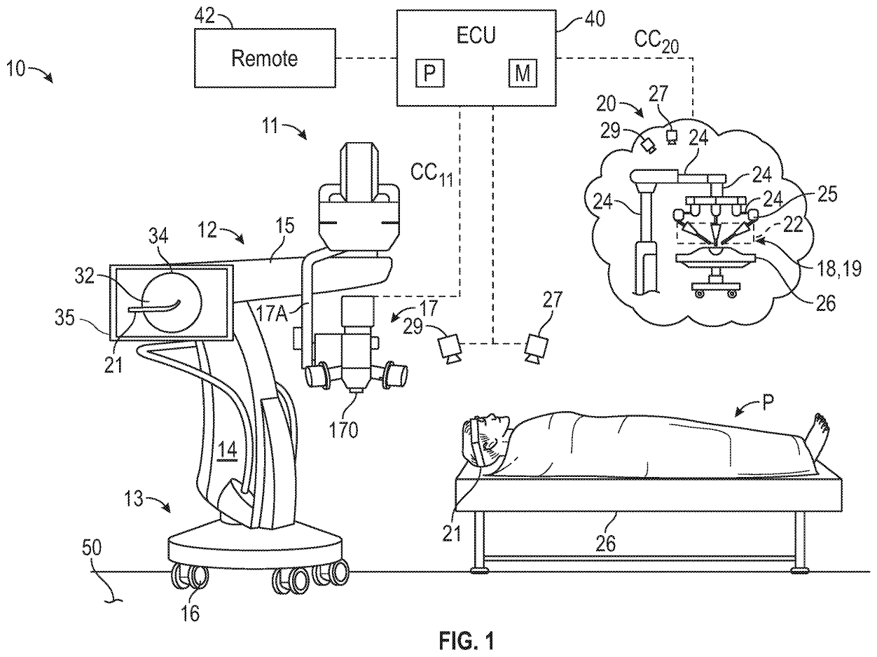

Referring to the drawings, wherein like reference numbers refer to like components, a representative ophthalmic surgical suite 10 is illustrated in FIG. 1. The ophthalmic surgical suite 10 is equipped with a robotic system 11 having a multi-axis visualization robot 12 and an operating table 26. During performance of a vitreoretinal, cataract, or other ocular surgical procedure within the suite 10, a patient P may be positioned on an operating table 26 or on another suitable platform, with a surgeon (not shown) seated or standing nearby. Although omitted from FIG. 1 for the purpose of illustrative simplicity, respective heights of the operating table 26 may be adjusted with the assistance of automatic and/or manual knobs, levers, or foot pedals in a typical implementation.

In one example, the surgical suite 10 includes a visualization robot 12 having a base 13 mounted or positioned relative to a floor 50 of the ophthalmic surgical suite 10, e.g., directly or via a mobile platform having lockable wheels 16 as shown. The base 13 in the illustrated exemplary embodiment of FIG. 1 is connected to a selective-compliance articulated robot arm (SCARA) 15 via an intervening support column 14. In general, the SCARA 15 is configured as an articulated serial robot arm used to support visualization equipment, such as a digital ophthalmic microscope 17 with an optical head 170, on a camera support arm 17A preparatory to or in conjunction with eye surgeries when performed within the suite 10. Utilizing associated hardware and software, the surgeon is able to view highly magnified images 34 of an eye 32 via a display screen 35 after proper positioning of the optical head 170.

As appreciated in the art, an ophthalmic microscope, such as the microscope 17 illustrated in FIG. 1, is comprised of several main components. The optical head 170 contains the various lenses and optics for magnifying and illuminating a patient's eye during a given procedure. Although omitted from FIG. 1 for illustrative simplicity, the optical head 170 may contain an objective lens and zoom section providing different magnifications, a pair of eyepieces through which the surgeon views magnified images of the eye, and a controllable light source.

Also present within the exemplary ophthalmic surgical suite 10 of FIG. 1 is an electronic control unit (ECU) 40. The ECU 40, e.g., one or more computer devices equipped with a non-transitory computer-readable storage medium, processors, and other application-suitable hardware and software, is typically configured for coordinating electronic features and settings of the optical head 170 and/or other equipment or payloads used within the suite 10, e.g., foot pedals, hand controls, filters, video cameras, beam splitters, etc. Control of the robotic system 11 via the ECU 40 occurs by transmitting electronic control signals (CC₁₁) to one or more actuators in the robotic system. In one example, the ECU 40 can include a graphics processing unit (GPU) or an edge device to aid in performing the image process discussed in the method 100 discussed below. Furthermore, the method 100 could be performed at a remote location 42 having a computer unit that is connected to the ECU 40 through a network connection.

Furthermore, as shown in FIG. 1, a robotic system 20 may be used in place of or in addition to the robotic system 11. In the illustrated example, the robotic system 20 includes serial arms/serial kinematics, parallel kinematics, or hybrid parallel-serial kinematics in different embodiments. The robotic system 20 is in communication with the ECU 40, for instance via hardwired transfer conductors and/or wireless pathways, such that the robotic system 20 is remotely controlled by electronic control signals (CC₂₀) from the ECU 40.

During performance of vitreoretinal, cataract, glaucoma, and other ocular surgeries, a patient may be positioned on an operating table 26 or on another suitable surgical stretcher, with the surgeon (not shown) positioned close to the patient's head, with remote station placement also being possible within the scope of the disclosure. Respective heights of the operating table 26 may be adjusted with the assistance of automatic and/or manual knobs, levers, or foot pedals (not shown) in a typical implementation. The surgeon may view magnified images of the eye 32 using the microscope 17, e.g., through analog or digital oculars or on a heads-up display using polarized three-dimensional (3D) glasses (not shown) or an auto-stereoscopic display.

The robotic system 20 also includes end-effector(s) 22 that may be securely mounted, for instance to the operating table 26 on which the patient P rests during the ocular surgery. The robot system 20 may include six or more links 24 and six or more electric motors/rotary actuators (A) 25 connected to the links and controllable via the ECU 40. The actuators 25 may be configured as slotless brushless direct current (BLDC) motors operable for positioning and/or actuating the surgical tools 18 and/or 19. The robotic system 20 also includes one or more sine-cosine position encoders operable for measuring/sensing an angular position of the slotless BLDC motors and for performing motor commutation thereof. Use of slotless BLDC motors herein is intended to minimize torque ripple in the autonomous (robotic system 20) implementation.

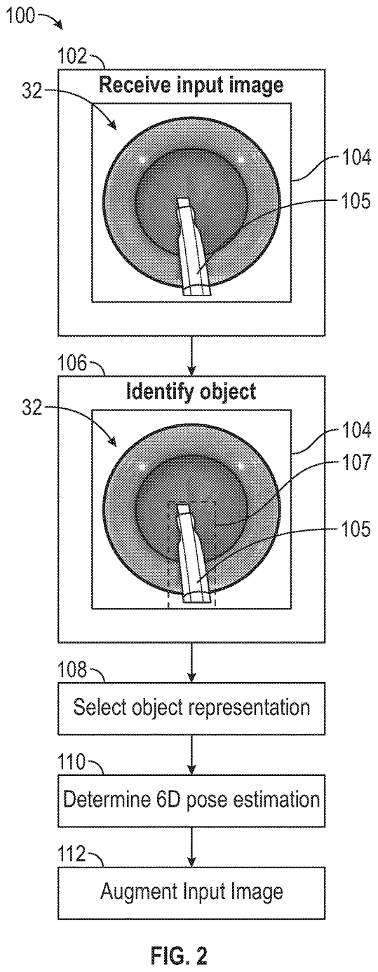

FIG. 2 illustrates a flow diagram of a method 100 of performing a six degree-of-freedom (6D) pose estimation for an object, such as one of the tools 18, 19, and 21 or the end-effectors 22, in an ophthalmic procedure. At block 102 (“Receive Input Image”), the method 100 begins by receiving an input image 104 of the ophthalmic surgical environment. In one example, the input image 104 is received from one or more sensors, such as the microscope 17, an optical camera 27, or a distance sensor 29, as shown in FIG. 1. When one of the optical sensors are used in connection with the distance sensor, the distance information can be fused or correlated to the input image 104 from the optical sensor to provide distance information for objects in the surgical environment. The distance information for the object(s) aids in determining the 6D pose estimation as discussed in greater detail below. With the input image 104 received, the method 100 proceeds to block 106.

At block 106 (“Identify Object”), the method 100 identifies an object 105 in the input image 104. The object 105 can include one or more of the tools 18, 19, and 21 or the end-effector(s) 22 of the robotic system 20. In one example, the object 105 is identified utilizing a bounding box 107 applied to the input image 104 to highlight a region of interest including the object 105. The bounding box 107 can be applied by a user manually selecting a region in the input image 104 with the object 105. Alternatively, the object 105 can be identified and highlighted with a machine learning model performing object detection. With the object 105 identified, the method 100 proceeds to block 108.

At block 108 (“Select Object Representation”), the method 100 selects an object representation that corresponds to the object 105 identified in the input image 104. The object representation provides a description of the object 105, such as a 3D representation of the object. In one example, the object representation is stored in the memory M and accessed by the processor P of the ECU 40 when the object 105 is identified at block 106. The object representation stored in the memory M can include a 3D computer model representing a mesh of the object 105, such as a computer aided drawing. Additionally, the object representation can include dimensions, such as an overall length, width, and depth, of the object 105 without providing a full 3D model of the object 105. The object representation can also be transferred to the ECU 40 through a wired or wireless network connection from a database located remote 42 of the ECU 40.

If the mesh of the object 105 is not available in the memory M or the remote location 42, the method 100 can generate the object representation. In one example, the method 100 generates the object representation by receiving a series input images 104 illustrating the object 105 in different 3D rotational positions and 3D translational positions relative to at least one of the optical sensors, such as the microscope 17 and/or the optical camera 27. Additionally, the distance sensor 29 can obtain measurements of the object 105 in the input images 104 based on a known relative position and field of view between the distance sensor 29 and the optical sensors, such as the microscope 17 and/or the optical camera 27.

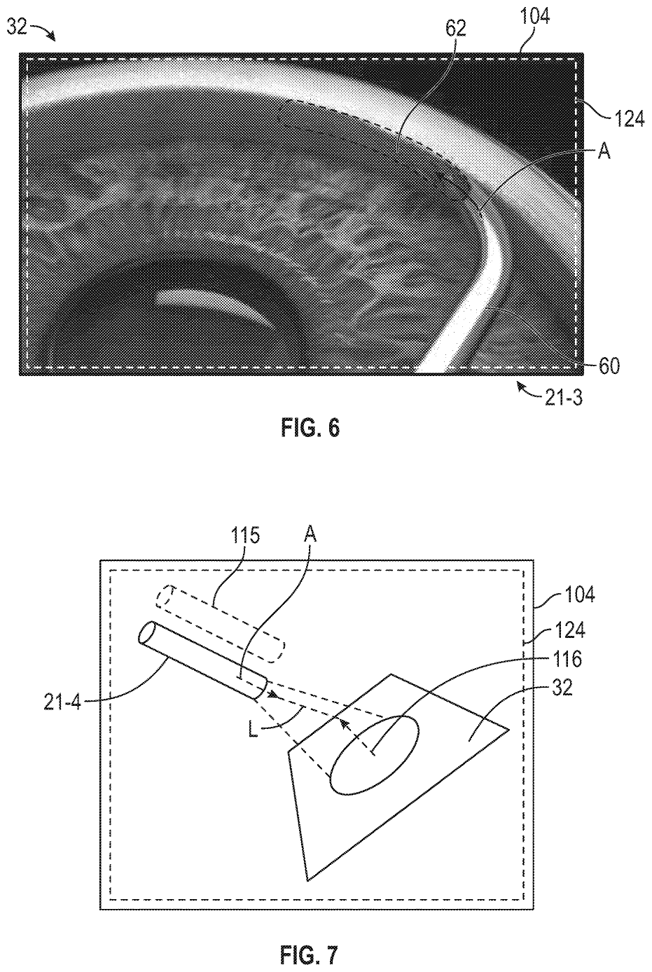

Depending on a type of the object 105, an additional set of input images 104 may capture the object 105 in different retracted or extended positions, such as with a tool 21-3 shown in FIG. 6. In some embodiments, the different retracted or extended positions can include predetermined retracted and/or extended positions representative of common surgical postures, e.g. a tool being inserted substantially orthogonal to the opening of a cannula, an insertion tool substantially parallel to Schlemm's canal, etc. The tool 21-3 includes an inserter 60 having a cannula with an opening at a distal end for positioning a stent 62 in the eye 32 during a minimally invasive glaucoma surgery (MIGS). The method 100 can also utilize finite element analysis (FEA) with ECU 40 to determine or predict how the stent 62 will move within the eye 32 when being inserted from the cannula.

Furthermore, a third set of input images 104 may capture the object 105, such as a tool 21-4 having a laser shown in FIG. 7, for performing laser coagulation. With the tool 21-4 configured to operate under different power intensities for emitting laser beams L at various levels of illumination. In one example, because the tool 21-4 can operate at different levels of illumination, the input images 104 can include the object 105 operating at a predetermined number of levels of illumination. This allows the method 100 to actively learn how the laser beams will behave. As discussed in greater detail below, this allows the method 100 to augment the input image 104 to predict a place of contact of the laser beams the surgical procedure. With the object representation obtained at block 108 with at least one of the above approaches, the method 100 proceeds to block 110.

At block 110 (“Determine 6D Pose Estimation”), the method 100 determines the 6D pose estimation for the object 105 identified in the input image(s) 104. The 6D pose estimation describes the object 105 identified in block 106 relative to its pose in six degrees of freedom (DOF). In one example, the 6D pose is described in terms of translational coordinates and rotational coordinates relative to a given reference frame, such as relative to one or more sensors, such as the microscope 17, or the optical camera 27. The translational coordinates describe the object 105 in three degrees of freedom utilizing an x-coordinate along an x-axis, a y-coordinate along a y-axis, and a z-coordinate along a z-axis. The rotational coordinates describe the object 105 in three additional degrees of freedom utilizing a roll around the x-axis, a pitch around the y-axis, and a yaw around the z-axis. In this disclosure, the “pose” of an object shall not connote a meaning of posing for “effect” (i.e. “posing for a photograph”); rather, an object's “pose” means the position of the object defined in the relevant reference frame.

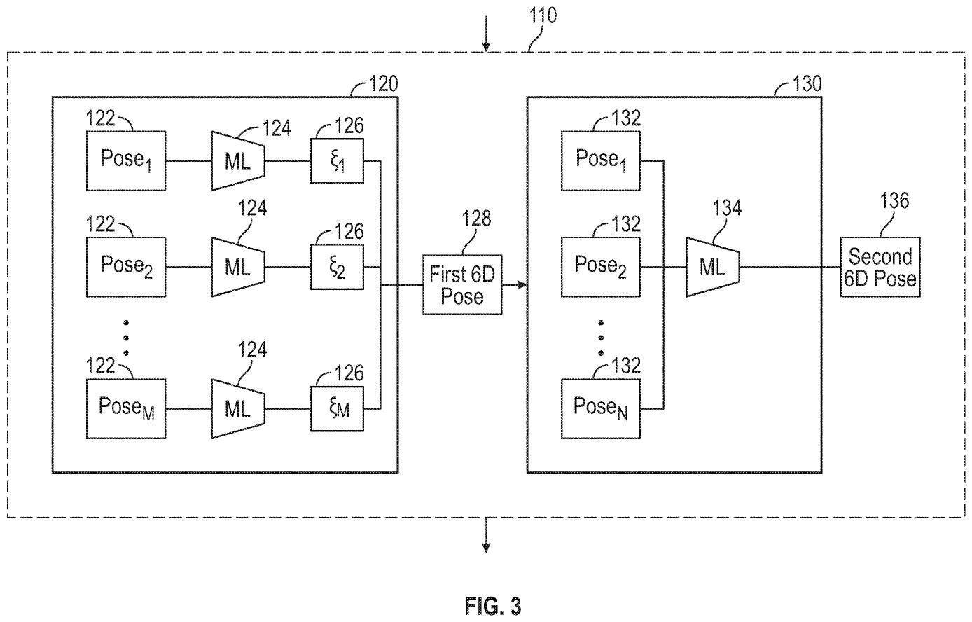

In one example, the method 100 determines the 6D pose estimation for the object 105 in the input image 104 by matching a pose of the object representation with the object 105 in the input image 104. FIG. 3 illustrates an expanded flow diagram for determining the 6D pose estimation from block 110 of FIG. 2. As shown in the example flow diagram of FIG. 3, determining the 6D pose estimation can be performed in one or more phases, such as a first or coarse estimation phase 120 followed by a second or refined estimation phase 130. One feature of utilizing two separate estimation phases for determining the 6D pose estimation of the object 105, is a reduction in a total number of processes performed to reach a final 6D pose estimation. The reduction in processes can be on the ECU 40 or with a computer at the remote location 42.

The reduction in the number of processes performed by the ECU or remote computer occurs by utilizing a first or coarse estimation phase 120 to select a first or coarse 6D pose estimation 128 and then performing a second or refined estimation phase 130 to select a second or refined 6D pose estimation 136. This approach allows the method 100 to reduce the number of 6D poses generated from the 3D representation selected at block 108 corresponding to the object 105 identified in the input image 104. While the illustrated example of FIG. 3 utilizes the first and second estimation phases 120 and 130, this disclosure applies to utilizing more than two or only a single estimation phase. The number of estimation phases utilized can depend on the available computer power of the ECU 40 or remote location or a desired level of accuracy for the 6D pose estimation performed by the method 100.

The first or coarse estimation phase 120 begins by generating a first set of 6D pose images 122 for the object representation from block 108. The first set of 6D pose images 122 are generated by manipulating the object representation from block 108 into different longitudinal and rotational positions for each image. The longitudinal and rotational positions correspond to coordinates relative to the x-axis, y-axis, and z-axis describing the object representation in three-dimensional space. In one example, the first set of 6D pose images 122, such as Pose₁, Pose₂, through Pose_{M}, include a first level of coarseness or variation in the longitudinal or rotational positions between adjacent images in a series of generated images. For example, the method 100 can generate an image of object representation centered at a given longitudinal position relative to one of the sensors and then selectively vary one or more of the roll, pitch, or yaw for the object representation. Furthermore, the roll, pitch, and yaw can be varied in equal increments, such as in five or ten degree increments, around 360 degrees of their respective axes. Furthermore, if the distance sensor 29 is available, it can be used to reduce a range of longitudinal positions for the first set of 6D pose images 122 be providing a distance estimate to the distance sensor 29 that can be correlated to one of the optical sensors, such as the microscope 17 and/or the optical camera 27.

With the first set of 6D pose images 122 generated, the images are then compared to the object 105 in the input image 104. In one example, the comparison is performed with a machine learning model to determine a first variation (ξ) 126. The first variation quantifies a difference between the object 105 in the input image 104 and the object representation in corresponding one of the first set of 6D pose images 122.

The method 100 selects a match from the first set of 6D pose images 122 for the object 105. In one example, the match includes the lowest variation 126 from the object 105 as determined by the machine learning model. With the first 6D pose estimation 128 determined by the first estimation phase 120, the method 100 then proceeds to the second estimation phase 130.

The second estimation phase 130 begins by generating a second set of 6D pose images 132 with the object representation at block 108. The second set of 6D pose images 132 are generated by manipulating the object representation from block 108 into different longitudinal and rotational positions for each image. In some embodiments, the second set of 6D pose images 132 includes a lesser number of images than the first set of 6D pose images 122, thereby reducing computational load during refinement. In one example, the second set of 6D pose images 132, such as Pose₁, Pose₂, through Pose_{N}, include a second level of coarseness or variation in the longitudinal or rotational positions between adjacent images in a series of generated images.

However, unlike the first set of 6D pose images 122, the second set of 6D pose images 132 do not include rotational variations surrounding 360 degrees for each of the axes. Rather, the second set of 6D pose images 132 are centered around the 6D coordinates of the first 6D pose estimation 128. The second set of 6D pose images 132 includes variations in roll, pitch, and yaw in smaller increments than the first set of 6D pose images 122 and within a predetermined range of the coordinates of the first 6D pose estimation 128 (e.g., may use a second level of coarseness or variation in that is smaller than the first level of coarseness). In one example, the predetermined range for the roll, pitch, and yaw is within one or two increments used to generate the first set of 6D pose images 122.

With the second set of generated 6D pose images 132 generated, the images are then compared to the object 105 in the input image 104. The method 100 selects a match from the second set of 6D pose images 132 for the object 105. In one example, the match is selected by a comparison performed with the machine learning model from the first estimation phase 120 to identify one of the second set of generated 6D pose images 132 with the least variation to the object 105 identified in the input image 104. The match then becomes the second or refined 6D pose estimation 136 such that the method 100 can utilize the coordinates of the object representation in the second 6D pose estimation 136 for creating an updated 6D pose estimation and augmenting the input image 104 as discussed in greater detail below.

In one example, the machine learning model used in block 110 is trained utilizing a training dataset that includes the object representation selected at block 108 presented in a predetermined number of known longitudinal and rotational positions. The training dataset is then used in connection with a machine learning algorithm to generate the machine learning model to select the 6D pose estimations with the least variation from the object 105 in the input image 104.

Furthermore, because the object 105 can be at least partially blocked or occluded by other structures in the input image 104, the machine learning model in block 110 is also trained to identify the object 105 under partially blocked or occluded scenarios. In these scenarios, the training dataset including a full view of the object 105 is modified to occlude a portion of the object 105 in the training images to generate a modified training dataset. In one example, the method 100 utilizes patch masking to modify the training dataset to occlude portions of the object 105 when generating the modified training dataset.

The machine learning algorithm can then use modified training dataset in connection with the training dataset as a ground truth to train the machine learning model to identify the object 105 when at least partially occluded in the input image 104. This also allows the method 100 to determine a location of the object 105 in the occluded portion of the input image 104 as discussed in greater detail below.

With the 6D pose estimation for the object 105 in the input image 104 selected as discussed above, the method 100 proceeds to block 112. At block 112 (“Augment Input Image”), the method 100 utilizes the 6D pose estimation for the object 105 from block 110 to augment the input image 104. In particular, the method utilizes the 6D coordinates of the object representation in the 6D pose estimation to determine a location and orientation of the object 105 in the input image 104.

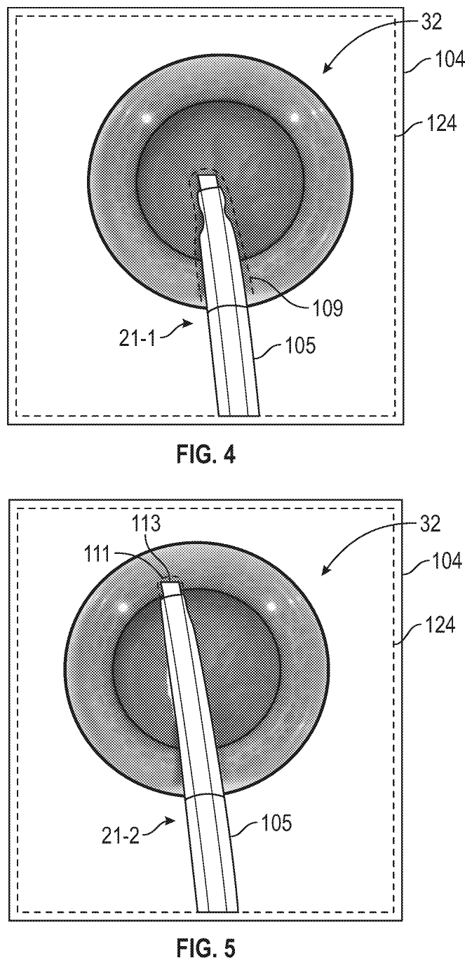

The method 100 utilizes the 6D pose estimation under several different approaches to augment the input image 104 depending on the type of ophthalmic procedure or environment captured in the input image 104. In the example shown in FIG. 4, the input image 104 includes a tool 21-1, such as a phaco-tip, captured in low lighting conditions. The low lighting conditions may make it difficult to visualize a boundary of the tool 21-1 and the anatomy of the eye. The method 100 can augment the image 104 by generating an image overlay 124 to highlight a perimeter of the object 105, such as with an image overlay 124 having dashed lines 109 that correspond to a perimeter of the tool 21-1. In one example, the dashed lines 109 can be of a contrasting color from the surrounding environment to improve visibility. The method 100 can identify the perimeter based on the 6D coordinates of the object representation in the 6D pose estimation corresponding to the object 105.

In another example, the method 100 utilizes its ability to identify the object 105 in the input images 104 when the object 105 is at least partially occluded. With the 6D pose estimation from block 110, the method 100 generates an image overlay 124 illustrating a location of the occluded portion of a tool 21-2, such as an irrigation and aspiration tip, as shown in FIG. 5. In one example, the location of the occluded portion of the object 105 is illustrated by generating an image overlay 124 including a dashed boundary line 111 corresponding to a location of a perimeter of the occluded portion of the object 105. In another example, the image overlay 124 can include a transparent representation 113 of the object representation based on the 6D pose estimation to illustrate the occluded portion of the object 105. In particular, this allows the user or surgeon to quickly determine an orientation or direction of a tip or occluded portion of the object 105.

In yet another example, the input image 104 is augmented by providing directional guidance to the user or surgeon to provide directions for moving the object 105 into a desired position or orientation. As shown in FIGS. 6-7, a directional arrow A can be applied through the image overlay 124 to direct the object 105, such as tool 21-3 or 21-4, to a desired location as shown in FIG. 6 or illustrate a location where light beams emitted from the object 105, such as a tool 21-4, will interact with a portion of the eye 32. The method 100 can also augment that input image 104 with virtual objects through the image overlay 124, such as path markers 115 or reflective directions 116, for aiding a surgeon during an operation.

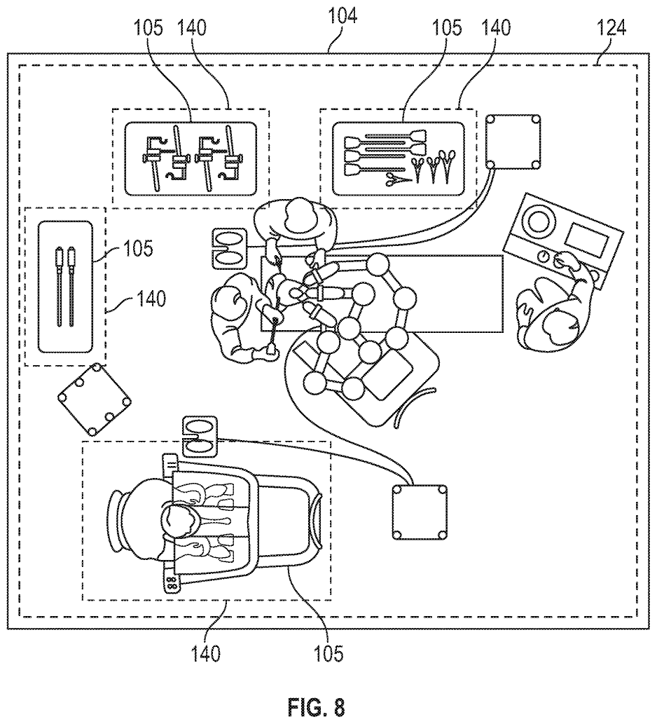

In yet another example, the input image 104 could include an ophthalmic surgical suite (FIG. 8) having multiple objects 105 for the method 100 to determine separate 6D pose estimations. One feature of having the method 100 determine separate 6D pose estimations for multiple pieces of surgical equipment in the surgical suite, is to create a human-object interaction map. This map can determine the patterns of personnel in the surgical suite. With the patterns of the personnel determined, the method 100 can provide recommendations to improve efficiency by reducing a distance between objects or rearranging the objects 105 in the surgical suite. Furthermore, the method 100 can determine when one or more of the objects 105 are outside of a predetermined perimeter 140 and provide an alert to the personnel in the surgical suite that one of the objects 105 needs to be repositioned or may be missing from the surgical suite.

Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

We claim:

1. A method of augmenting an image from an ophthalmic surgical suite, the method comprising:
receiving an input image from the ophthalmic surgical suite from at least one sensor;
identifying an object in the input image;
selecting an object representation corresponding to the object identified in the input image;
determining a 6D pose estimation for the object in the input image by:
creating a first set of generated 6D pose images each illustrating the object representation in one of a plurality of first coordinates, wherein the first coordinates each define a longitudinal and rotational position for the object representation;
identifying a first match between the first set of generated 6D pose images and the object in the input image, wherein the first match includes the least variation between the object and the corresponding one of the first set of generated 6D pose images; and
selecting the 6D pose estimation for the object based on coordinates of the first match; and
augmenting the input image based on the 6D pose estimation.

2. The method of claim 1, wherein estimating the 6D pose for the object in the input image includes:
creating a second set of generated 6D pose images each illustrating the object representation in one of a plurality of second coordinates, wherein the second coordinates each define a longitudinal and rotational position for the object representation;
identifying a second match between the second set of generated 6D pose images and the object in the input image, wherein the second match includes the least variation from the object and the corresponding one of the second set of generated 6D pose images; and
updating the 6D pose estimation for the object based on coordinates of the second match.

3. The method of claim 2, wherein the first plurality of coordinates includes a first level of coarseness between at least one of longitudinal and rotational positions and the second plurality of coordinates includes a second level of coarseness between at least one of longitudinal and rotational positions that is less than the first level of coarseness.

4. The method of claim 3, wherein the first set of generated 6D pose images includes a greater number of images than the second set of generated 6D pose images.

5. The method of claim 3, wherein the second plurality of coordinates are within a predetermined range of rotational positions from the first match.

6. The method of claim 5, wherein the second plurality of coordinates are within a predetermined range of longitudinal positions from the first match.

7. The method of claim 1, wherein augmenting the input image includes generating directional markers guiding the object to a desired location within the input image during an ophthalmic procedure.

8. The method of claim 1, wherein a machine learning model is utilized to identify the first match from the first set of generated 6D pose images with the object identified in the input image.

9. The method of claim 8, including training the machine learning model to identify an occluded portion of the object in the input image by:
generating a training dataset including the object representation at a predetermined number of coordinates with known translational and rotational positions;
modifying the training dataset to occlude a portion of the object representation from corresponding images in the training dataset to generate a modified training dataset; and
utilizing the modified training dataset with the training dataset as a ground truth in connection with a machine learning algorithm to train the machine learning model to identify the occluded portion of the object in the input image.

10. The method of claim 9, wherein augmenting the input image includes identifying an occluded portion of the object in the input image and highlighting the occluded portion of the object in the input image.

11. The method of claim 10, wherein highlighting the occluded portion of the object in the input image includes generating an image overlay having a dashed line surrounding a perimeter of the occluded portion.

12. The method of claim 10, wherein highlighting the occluded portion of the object in the input image includes generating an image overlay of the object representation based on the 6D pose estimation corresponding to a region of the occluded portion.

13. The method of claim 1, wherein the object representation is generated based on a plurality of input images illustrating the object in a plurality of different longitudinal and rotational positions relative to the at least one sensor.

14. The method of claim 1, wherein the object representation is generated based on a plurality of input images illustrating an illumination intensity of the object at a plurality of different levels of intensity.

15. The method of claim 1, wherein the input image illustrates an ophthalmic surgical suite and the object includes at least one piece of surgical equipment within the ophthalmic surgical suite and the method includes generating a notification if the 6D pose estimation for the at least one piece of surgical equipment is located outside of a predetermined region in the ophthalmic surgical suite.

16. A system for visualizing an object in an ophthalmic surgical suite, the system comprising:
a single optical sensor configured to obtain an input image from an ophthalmic procedure;
a controller in communication with the single optical sensor, wherein the controller is configured to:
receive an input image from the ophthalmic surgical suite from at least one sensor;
identify an object in the input image;
select an object representation corresponding to the object identified in the input image;
determine a 6D pose estimation for the object in the input image by:
creating a first set of generated 6D pose images each illustrating the object representation in one of a plurality of first coordinates, wherein the first coordinates each define a longitudinal and rotational position for the object representation;
identifying a first match between the first set of generated 6D pose images and the object in the input image, wherein the first match includes the least variation between the object and the corresponding one of the first set of generated 6D pose images; and
selecting the 6D pose estimation for the object based on coordinates of the first match; and
augment the input image based on the 6D pose estimation.

17. The system of claim 16, wherein the controller is configured to determine the 6D pose estimation for the object in the input image by:
creating a second set of generated 6D pose images each illustrating the object representation in one of a plurality of second coordinates, wherein the second coordinates each define a longitudinal and rotational position for the object representation;
identifying a second match between the second set of generated 6D pose images and the object in the input image, wherein the second match includes the least variation from the object and the corresponding one of the second set of generated 6D pose images; and
updating the 6D pose estimation for the object based on coordinates of the second match.

18. A non-transitory computer-readable medium embodying programmed instructions which, when executed by a processor, are operable for performing a method comprising:
receiving an input image of an ophthalmic surgical suite from at least one sensor;
identifying an object in the input image;
selecting an object representation corresponding to the object identified in the input image;
determining a 6D pose estimation for the object in the input image by:
creating a first set of generated 6D pose images each illustrating the object representation in one of a plurality of first coordinates, wherein the first coordinates each define a longitudinal and rotational position for the object representation;
identifying a first match between the first set of generated 6D pose images and the object in the input image, wherein the first match includes the least variation between the object and the corresponding one of the first set of generated 6D pose images; and
selecting the 6D pose estimation for the object based on coordinates of the first match; and
augmenting the input image based on the 6D pose estimation.

19. The non-transitory computer-readable medium of claim 18, wherein a machine learning model is utilized for comparing the first set of generated 6D pose images to the object identified in the input image, wherein the machine learning model is trained by:
generating a training dataset including the object representation at a predetermined number of coordinates with known translational and rotational positions; and
utilizing the training dataset in connection with a machine learning algorithm to generate the machine learning model to determine the first set of generated 6D pose images with the least variation from the object in the input image.

20. The non-transitory computer-readable medium of claim 19, including training the machine learning model to identify occluded portions of the object in the input image by:
modifying the training dataset to occlude a portion of the object representation from corresponding images in the training dataset to generate a modified training dataset; and
utilizing the modified training dataset with the training dataset as a ground truth in connection with the machine learning algorithm to train the machine learning model to identify the occluded portion of the object in the input image.
